(19) United States
(12) Patent Application Publication
(10) Pub. No.: US 2015/0051530 A1
(43) Pub. Date: Feb. 19, 2015

(54) DESIGNS FOR WOUND SUPPORT APPARATUS

(71) Applicant: Insightra Medical Inc., Irvine, CA (US)

(72) Inventors: Wayne A. Noda, Mission Viejo, CA (US); Bradley Sharp, Irvine, CA (US); Stephen Graham Bell, Rome (IT); Lloyd Lowry, Irvine, CA (US); Daniel Hyman, Irvine, CA (US); John Acker, Irvine, CA (US)

(21) Appl. No.: 14/451,706

(22) Filed: Aug. 5, 2014

Related U.S. Application Data
(60) Provisional application No. 61/867,099, filed on Aug. 18, 2013.

(51) Int. Cl. A61B 17/08

(52) U.S. Cl. CPC A61B 17/08; A61B 17/085; A61B 2017/081
USPC 602/41

(57) ABSTRACT

A connector for holding wound closure straps in tension to close a wound. The connector may be a quick-release connector. It may include a tension indicator. The connector may have a magnetic coupling, elastic portion, or arcuate flexure joining the strap-engaging ends of the connector.

Priority is claimed from U.S. provisional patent application 61/867,099, filed Aug. 18, 2013.

### I. FIELD OF THE INVENTION

This application is generally related to wound support apparatus.

### II. BACKGROUND OF THE INVENTION

In the field of wound management, there is a need to gradually approximate the skin to achieve closure of an open wound. These can be a result of pressure sores and ulcers, diabetes, amputations, trauma, cancer, burns, etc. By applying a constant force on the skin adjacent to the wound, the skin will gradually migrate and heal over the wound. This process occurs slowly over many days.

### SUMMARY OF THE INVENTION

Present principles recognize that wound patients often are elderly or diabetic or suffering from other morbidities that leaves the skin friable and easily injured. As understood herein, it is consequently desirable not to apply excessive forces onto the skin.

Accordingly, an assembly includes a first strap arrangement having a first end configured for engaging skin of a patient and a second end. A first strap body extends between the ends of the first strap arrangement A second strap arrangement has a first end configured for engaging skin of a patient and a second end, and a second strap body extends between the ends of the second strap arrangement. A quick release buckle is configured for engaging the strap bodies to hold the strap bodies in tension.

In some implementations the quick release buckle has first and second apertures separated from each other by a central wall, with each aperture being defined at least in part by the central wall and a respective outer wall spaced from the central wall and generally parallel thereto. At least one of the outer walls is discontinuous to form a passageway through which a strap body can be passed to engage and disengage the strap with the buckle. If desired, both outer walls can be discontinuous and can form respective passageways. In this embodiment, the buckle is flexible such that a person can compress a first one of the discontinuous outer walls to thereby enlarge the passageway formed by a second one of the outer walls.

In other examples, the buckle has a female element with an open end and side slots and a male element with opposed resilient arms. The tangs of the male element are receivable through the open end of the female element and each tang is exposable through a respective slot, so that a person can slide the male element into the female element and the tangs engage the slots to hold the elements together, and so that a person can press the tangs exposed in the respective slots toward each other and pull the elements apart to disengage the elements.

The first ends of the respective strap arrangements may include respective pads engaged with the respective strap bodies, with each pad having an adhesive surface for adhering to the skin and an opposed receiving surface configured for engaging the respective second end of the respective strap arrangement. Or, the first ends of the respective strap arrangements can include respective pads configured to be held onto the skin by staples or sutures or subcutaneous anchors.

If desired, at least one end of at least one of the strap arrangements can include at least one visible reference line oriented perpendicularly to a long axis defined by the strap arrangement. In some examples given below, the assembly is a first assembly defining a long axis and engaged with opposed elongated footings oriented perpendicularly to the long axis, and the footings also are engage with at least a second assembly oriented parallel to and space from the first assembly. The footings can be configured to engage respective skin adhesive pads.

A wound dressing constituent may be disposed on the buckle. The buckle may be curved in longitudinal cross-section to establish a concave or convex bottom surface the faces the patient.

In another aspect, an assembly includes a first strap arrangement having a first end configured for engaging skin of a patient and a second end. A first strap body extends between the ends of the first strap arrangement A second strap arrangement has a first end configured for engaging skin of a patient and a second end, and a second strap body extends between the ends of the second strap arrangement. A connector is configured for engaging both strap bodies to hold the strap bodies in tension. The connector includes at least one tension indicator such as an (LED) that is energized responsive to a predetermined tension being reached, and/or a digital display presenting an alpha-numeric indication of tension, and/or a spring interconnecting opposed pieces of the connector in combination with a transverse hash mark on at least one of the pieces, and/or a wireless transceiver generating a wireless signal representative of tension.

In another aspect, an assembly includes a first strap arrangement having a first end configured for engaging skin of a patient and a second end. A first strap body extends between the ends of the first strap arrangement A second strap arrangement has a first end configured for engaging skin of a patient and a second end, and a second strap body extends between the ends of the second strap arrangement. A connector is configured for engaging both strap bodies to hold the strap bodies in tension. The connector includes a first end portion engaged with the first strap body, a second end portion engaged with the second strap body, and an interconnect connecting the first and second end portions. The interconnect is selected from the group of magnet, elastic band, arcuate flexure.

The details of the present application, both as to its structure and operation, can best be understood in reference to the accompanying drawings, in which like reference numerals refer to like parts, and in which:

### BRIEF DESCRIPTION OF THE DRAWINGS

FIG. 1 is a perspective view of a wound closure apparatus with two opposing strap assemblies, each with a base that can be adhered to the skin, joined with a buckle element;

FIG. 2 is a plan view of a buckle design that enables quick strap release by squeezing the strap material and releasing through the opening in the buckle slot;

FIGS. 3 and 3A are plan views of an alternate buckle with openings on both opposing slots;

FIG. 4 is a plan view showing a quick release snap lock buckle;

FIG. 5 is a plan view illustrating a buckle being positioned off center from a wound to prevent any irritation or discomfort that might be caused by the buckle assembly;

FIG. 6 is a plan view of a buckle with an indicator lamp that illuminates based on tension;

FIG. 7 is a plan view of an alternate tension-indicating buckle;

FIG. 8 is a plan view of yet another tension-indicating buckle assembly;

FIG. 9 is a plan view of portions of a wound closure strap assembly with a buckle and tension transmitter in the buckle;

FIGS. 10 and 10A are perspective views of portions of a wound closure strap assembly with a force indicator to set the straps at a predetermined and then replaced with a slide-in buckle;

FIG. 11 is a schematic diagram of an example non-limiting force indicating assembly;

FIG. 12 is a plan view of a buckle employing magnetic coupling to limit strap tension;

FIG. 13 is a plan view of a buckle assembly with a middle element made of elastomeric material;

FIG. 14 is an elevation view of portions of a wound closure strap assembly with the straps being joined by an arched, shaped spring;

FIG. 15 is a plan view of a wound closure strap assembly with reference lines on the adhesive pads;

FIG. 16 illustrates portions of a wound closure strap being used as a means to mark and record closure progress over time;

FIG. 17 is a partial cross-sectional elevation view of a buckle assembly with a wound dressing;

FIG. 18 is a perspective view of portions of a wound closure strap assembly with an arcuate buckle the concave surface of which faces the wound;

FIG. 19 is a perspective view of portions of a wound closure strap assembly with an arcuate buckle the convex surface of which faces the wound;

FIG. 20 is a perspective view of portions of a wound closure strap assembly with straps having non-adhesive bases anchored to the patient using skin staples, subcutaneous anchors or sutures;

FIG. 21 is a top perspective view of a wound closure strap assembly with an adhesive anchor configured as a longer base strip;

FIG. 22 is a perspective view of a wound closure strap assembly with replaceable adhesive strips;

FIG. 23 is an exploded view of a wound closure strap assembly with a combination dressing and tensioning strap assembly; and

FIG. 24 is a perspective view of wound closure strap assembly with a body-conforming adhesive anchor base.

### DETAILED DESCRIPTION

FIG. 1 shows a wound closure assembly 10 that includes two opposing strap arrangements 12, 14, each with a base that can be adhered to the skin as described further below, joined with a buckle 16 that may be established by any one of the buckles or connectors divulged herein. The buckle 16 holds the strap assemblies 12, 14 in tension so that they pull their respective bases toward each to thereby also pull the skin portions to which the bases are attached toward each other, to close a wound between the skin portions.

With greater particularity, the first strap arrangement 12 has a first end 18 configured for engaging skin of a patient, a second end 20, and a first strap body 22 extending between the ends 18, 20 of the first strap arrangement 12. The second strap arrangement 14 may be substantially identical in configuration and construction to the first strap arrangement 12 as shown. In the example shown, the first end 18 is established by a pad that may be made integrally with the strap body 22 and may be wider than the strap body 22. The bottom surface 24 of the pad typically has biocompatible adhesive deposited on it so that when pressed onto the skin of the patient, the bottom surface 24 sticks to the patient. On the other hand, the top surface 26 may have loop fasteners 28 disposed on it to engage complementarily-shape hook fasteners 30 on the second end 20, which, as shown, may be made integrally with the strap body 22 and which may be the same width as the strap body 22. The hooks and loops may be reversed. A trade name for a common type of hook and eye fastening mechanism is Velcro. The ends 18, 20 alternatively may be made separately from the strap body 22 and connected thereto, e.g., by sewing or other means.

It may now be appreciated that the first ends of the strap arrangements 12, 14 can be adhered to the skin on opposite sides of a wound, the strap bodies pulled through the buckle 16 and cinched to tension the strap bodies, and then the second ends placed onto the top surfaces of the first ends to hold the ends together with the strap bodies in tension to, in cooperation with each other, pull skin on opposite sides of the wound toward the wound.

In the example shown, the buckle 16 preferably is a quick-release buckle to facilitate quickly inserting and removing a strap body from the body, with the understanding that as wound closure progresses, it is typically necessary to re-adjust the tension of the straps and that this can entail disengaging and re-engaging the strap bodies with the buckle 16.

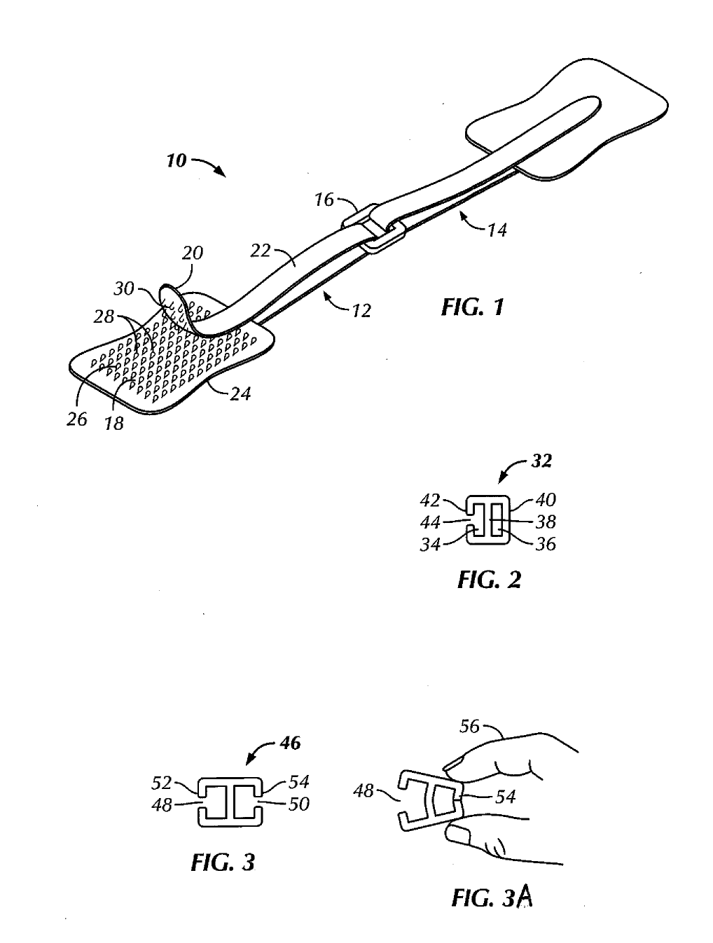

FIG. 2 shows an example quick release buckle 32 that enables quick strap release by squeezing the strap material and releasing through a passageway or opening in a buckle aperture. This feature can be beneficial for quick release during dressing changes.

With more particularity, the quick release buckle 32 of FIG. 2 has first and second apertures 34, 36 separated from each other by a central wall 38. Each aperture 34, 36 is defined at least in part by the central wall 38 and a respective outer wall 40, 42 that is spaced from the central wall 38 and that is generally parallel thereto as shown. While a quick-release buckle may have two opposed continuous outer walls, in the example shown, the left-most outer wall 42 is discontinuous to form a passageway 44 through which a strap body 22 can be passed to engage and disengage the strap with the buckle.

FIGS. 3 and 3A show a quick release buckle 46 with openings or passageways 48, 50 on both opposing outer walls 52, 54. The buckle 46 can be made from a thermoplastic elastomer with flexural properties that allows flexing without breakage. As shown in FIG. 3A, squeezing one end forces the opposite opening or passageway to open to easily release the strap body 22. That is, in the embodiment shown both outer walls 52, 54 are discontinuous and form respective passageways 48, 50. In this embodiment, the buckle is flexible such that a person 56 can compress a first one of the discontinuous outer walls 54 to thereby enlarge the passageway 48 formed by a second one of the outer walls 52.

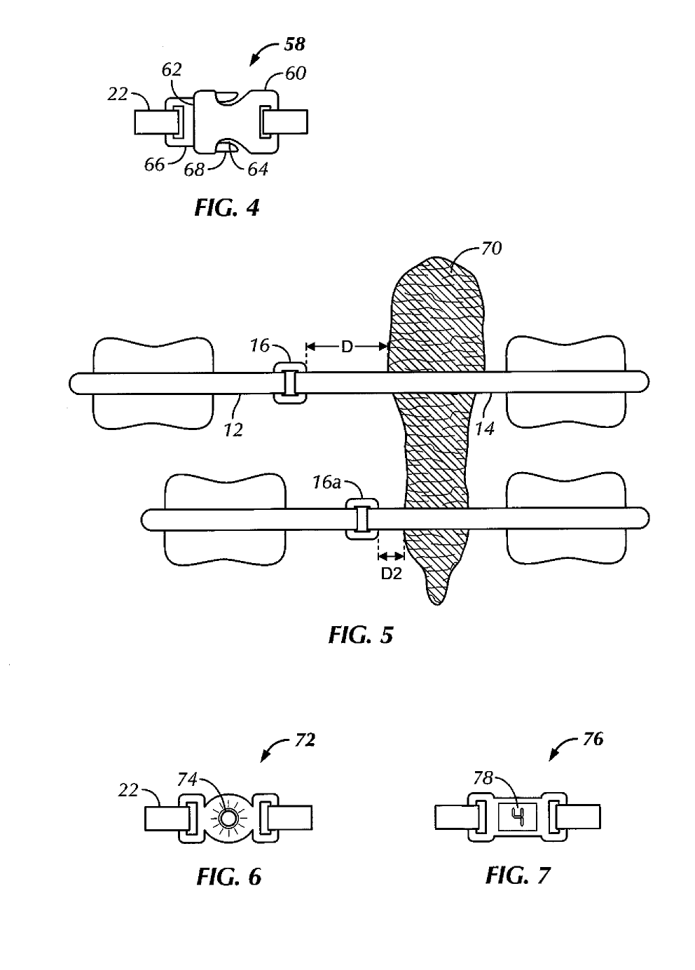

In the example shown in FIG. 4, a quick release buckle 58 has a female element 60 with an open end 62 and side slots 64. A male element 66 has opposed resilient tangs 68. The tangs 68 of the male element 66 are receivable through the open end 62 of the female element 60, and each tang 68 is exposed through a respective slot 64 when the male element is fully inserted into the female element, interlocking with the structure of the female element surrounding the slot. A person can slide the male element into the female element and the tangs engage the slots to hold the elements together, and a person can press the tangs exposed in the respective slots toward each other and pull the elements apart to disengage the elements.

Before proceeding with further buckle/connector features, reference is made briefly to FIG. 5. As shown, the buckle 16 is positioned off center from a wound 70, i.e., closer to the adhesive pad end of one strap arrangement 12 than to the adhesive pad of the other strap arrangement 14 to prevent any irritation or discomfort that might be caused by the buckle 16. The buckle 16 of the top assembly shown in FIG. 5 is positioned a distance “D” from the nearest edge of a relatively large part of the wound 70 as shown, whereas the buckle 16a of the bottom assembly shown in FIG. 5 is positioned a distance “D2” from the nearest edge of a relatively narrow part of the wound 70, with D2 being less than D.

FIGS. 6-10 illustrate various embodiments of a strap body connector configured for engaging both strap bodies to hold the strap bodies in tension which includes at least one tension indicator. Note that the tension indicators described herein may be used with any of the buckle configurations shown if desired and may also be used with conventional buckle/connector structures.

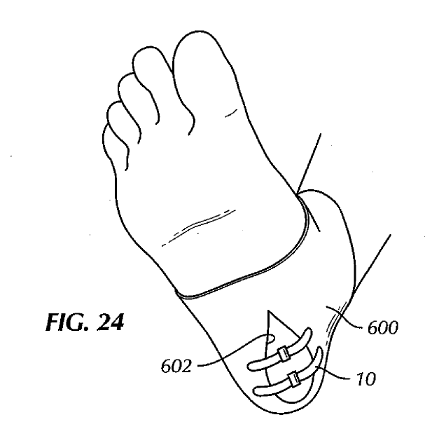

In the embodiment of FIG. 6, a strap connector 72 has a force sensing element within the connector. Example sensors are described below in reference to FIG. 24. When the tension sensed by the sensor satisfies a threshold, the signal from the sensor is used or processed as appropriate to illuminate a lamp 74. If desired, the threshold can be selected to activate the lamp 74 when a predetermined “proper” amount of tension is applied to the strap bodies 22. In some examples the lamp 74 may be established by a light emitting diode (LED) that is energized responsive to a predetermined tension being reached.

FIG. 7 shows a connector 76 with a tension sensor therein the signal of which is used to provide an alpha-numeric indication of tension in the strap assembly on a digital display 78, such as a small flat panel display. This enables an operator to gain insight into the actual numeric tension force in the strap assembly.

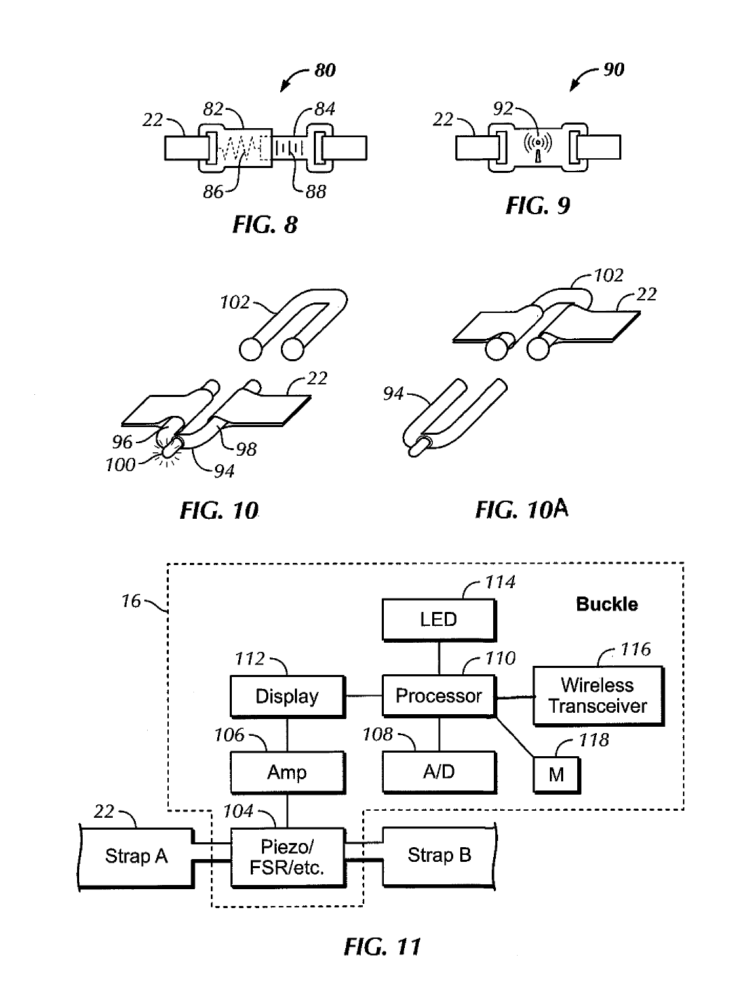

FIG. 8 shows a connector 80 that includes a hollow first piece 82 engaged with the first strap body 22 and a second piece 84 reciprocatingly engaged with the first piece 82 and engaged with the second strap body. While the pieces 82, 84 may be parallelepiped-shaped as shown, cylindrical or other shapes may be employed. A spring 86 (shown in phantom in FIG. 8) interconnects the pieces 82, 84, and the tension indicator includes at least one and preferably plural transverse hash marks 88 on one or both of the pieces (hash marks shown only on the second piece 84 in the example of FIG. 8). The spring 86 may be resistive to tension and may be connected to ends of the pieces 82, 84. It will readily be appreciated that the number of visible hash marks 88 indicates the amount of force based on how much the spring is being stretched.

In FIG. 9, a connector 90 has a tension sensor and a wireless transceiver 92 receiving signals from the sensor and generating a wireless signal representative of tension in response. The wireless signal may be sent to remote receiver such as a monitor or a smartphone app for presentation of an indication of the tension in the strap assembly by the receiver. This can be beneficial when the primary caretaker is not in the same location as the patient.

FIGS. 10 and 10A illustrate that a U-shaped connector 94 can define opposed parallel legs 96, 98 that can be respectively engaged with the loops formed by doubled-back strap bodies 22 according to principles above, with the strap bodies being cinched to impart tension in the assembly. A tension sensor in the connector 94 can, as described above, illuminate a lamp 100 when a predetermined tension is sensed. Upon illumination of the lamp 100, a second connector 102 without a tension sensor or lamp may be inserted into the loops (deforming the legs outwardly as necessary to engage them with the loops), and after the second connector 102 is inserted into the loops, the first connector 94 may be removed. This enables only one force measuring device-equipped connector to be needed to set all straps being used.

FIG. 11 illustrates a block diagram of a tension sensing system that may be incorporated in any of the connectors described (collectively labeled 16 in FIG. 11 for simplicity). The tension imparted by the strap bodies 22 is imparted to a tension sensor 104 such as a piezoelectric sensor, a force sensing resistor, or other force sensor. The signal may be amplified by an amplifier 106, digitized by an analog to digital converter 108, and provided to a processor 110. According to principles above, the processor 110 may output a digital representation of the tension on a display 112, may illuminate a lamp 114 responsive to a determination that the sensed tension satisfies a threshold, and/or cause a wireless transceiver 116 to send a signal representing the tension to a receiving device.

Attendant to FIG. 11, the processor 110 may access instructions on a computer memory 118. As used herein, instructions refer to computer-implemented steps for processing information in the system. Instructions can be implemented in software, firmware or hardware and include any type of programmed step undertaken by components of the system.

A processor may be any conventional general purpose single- or multi-chip processor that can execute logic by means of various lines such as address lines, data lines, and control lines and registers and shift registers.

Software modules described by way of the flow charts, user interfaces, or prose can include various sub-routines, procedures, etc. Without limiting the disclosure, logic stated to be executed by a particular module can be redistributed to other software modules and/or combined together in a single module and or made available in a shareable library.

Present principles described herein can be implemented as hardware, software, firmware, or combinations thereof; hence, illustrative components, blocks, modules, circuits, and steps are set forth in terms of their functionality.

Further to what has been alluded to above, logical blocks, modules, and circuits described below can be implemented or performed with a general purpose processor, a digital signal processor (DSP), a field programmable gate array (FPGA) or other programmable logic device such as an application specific integrated circuit (ASIC), discrete gate or transistor logic, discrete hardware components, or any combination thereof designed to perform the functions described herein. A processor can be implemented by a controller or state machine or a combination of computing devices.

The functions and methods described below, when implemented in software, can be written in an appropriate language such as but not limited to hypertext transfer protocol language (html)-5, C# or C++, and can be stored on or transmitted through a computer-readable storage medium such as a random access memory (RAM), read-only memory (ROM), electrically erasable programmable read-only memory (EEPROM), compact disk read-only memory (CD-ROM) or other optical disk storage such as digital versatile disc (DVD), magnetic disk storage or other magnetic storage devices including removable thumb drives, etc. A connection may establish a computer-readable medium. Such connections can include, as examples, hard-wired cables including fiber optics and coaxial wires and digital subscriber line (DSL) and twisted pair wires. Such connections may include wireless communication connections including infrared and radio.

The transceiver 116 may be, without limitation, a Bluetooth transceiver, a Wi-Fi transceiver, a wireless telephony transceiver, or other transceiver such as a mesh network transceiver.

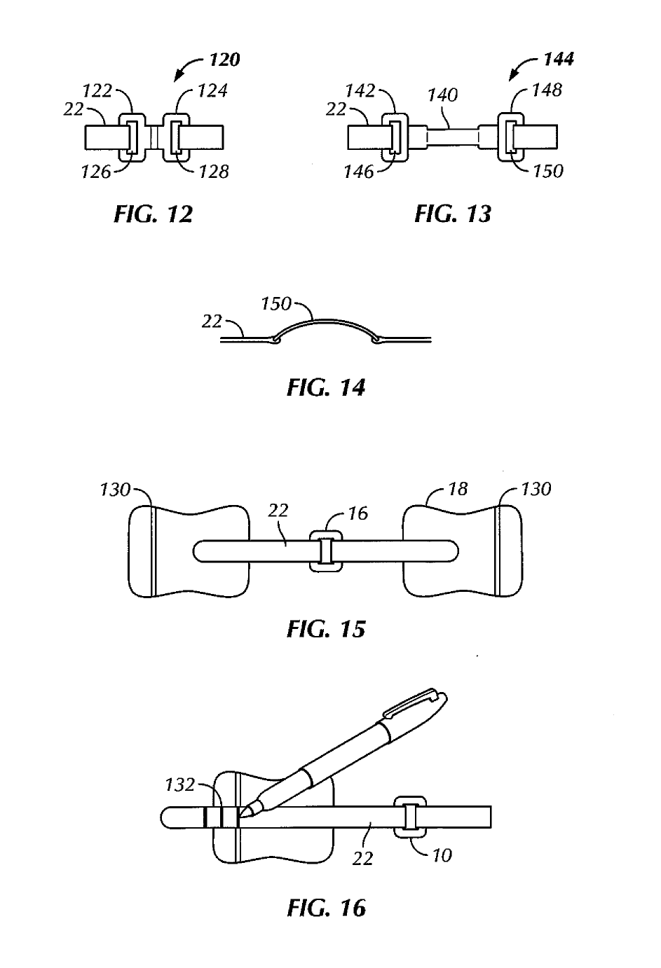

FIGS. 12, 13, and 14 illustrate strap body connectors with an interconnector. In FIG. 12, a connector 120 includes a first end portion 122 engaged with the first strap body 22, a second end portion 124 engaged with the second strap body, and a magnetic-based interconnector. In the example shown, the first end portion 122 defines a first aperture 126 through which the first strap body 22 extends, the second end portion 124 defines a second aperture 128 through which the second strap body extends, and the interconnect includes at least a first magnet 130 engaged with the first end portion 122 and at least a ferromagnetic element 132 engaged with the second end portion 124. The ferromagnetic element 132 may be another permanent magnet with its positive pole facing the negative pole of the magnet 130 (or vice-versa). The strength of the magnetic attraction between the magnet 130 and element 132 advantageously may be established based on a maximum tension force desired, such that the elements 130, 132 separate if that limit is exceeded.

FIG. 13 shows an alternate interconnect made of an elastomeric element 140 such as a piece of silastic tubing. This provides a shock protection feature as well as a more even and constant tensile force in between adjustments.

With greater particularity, a first end portion 142 of a connector 144 defines a first aperture 146 through which the first strap body 22 extends, and a second end portion 148 defining a second aperture through which the second strap body extends. An interconnect 140 includes an elastic band.

In FIG. 14, an interconnect 150 between first and second strap bodies 22 is established by an arcuate flexure having opposed first and second ends defining first and second end portions, respectively that are connected to the strap bodies 22. In FIG. 14, the strap bodies 22 are joined by an arched shaped spring such as tempered stainless steel. As the ends pull against the spring, a tensile force is generated. This will act as a constant spring force during the period between adjustments.

Referring to FIGS. 15 and 16, in FIG. 15 reference lines 130 may be printed or otherwise formed on one or both adhesive pad ends 18 so that the healing process can be measured and monitored over time. FIG. 16 shows a strap body 22 being used as a means to mark and record progress over time with reference lines 132. Each time the strap is adjusted, it is marked with respect to the reference line. The strap can be made from a translucent material to aide in visualizing the reference line below on the base.

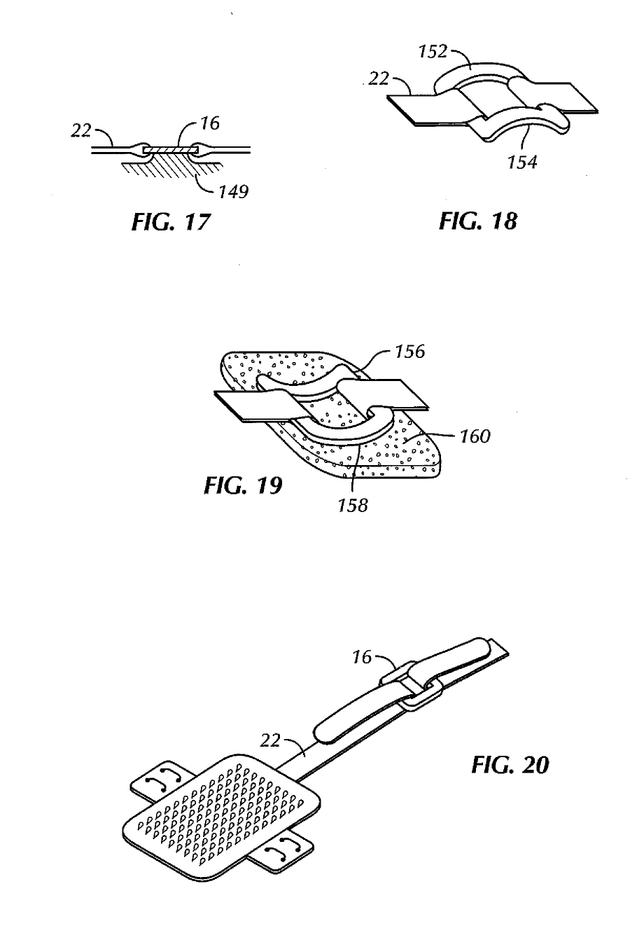

FIG. 17 shows any of the buckles herein in cross section which may be attached to a wound dressing 149. The dressing 149 can be of various wound treatments, colloid type containing moisture, drugs, antibacterial, etc.

It is recognized that the buckle, dressing, strap or the entire assembly can be comprised of materials that are beneficial to wound management. Antibacterial, drug eluting, infection detecting materials, growth factors, etc.

FIGS. 18 and 19 illustrate that any of the buckles herein may be arcuate in longitudinal cross-section. FIG. 18 shows a connector 152 that is formed with an arcuate shape. This prevents the buckle from coming in contact with the wound. In the embodiment shown in FIG. 18, the connector 152 has a concave surface 154 facing the skin.

In contrast, FIG. 19 shows a curved connector 156 faced downward toward the wound such that a convex surface 158 faces the skin. In some instances, it is desirable to apply pressure to the wound. The buckle and or the dressing 160 under it, can be sized and arced to impart the desired the amount of pressure. The buckle can be made from a malleable material such as aluminum so that it can be shaped as desired at the time of application.

FIG. 20 illustrates that the strap arrangements need not be adhesively attached to the skin, but can be anchored using skin staples, subcutaneous anchors or sutures.

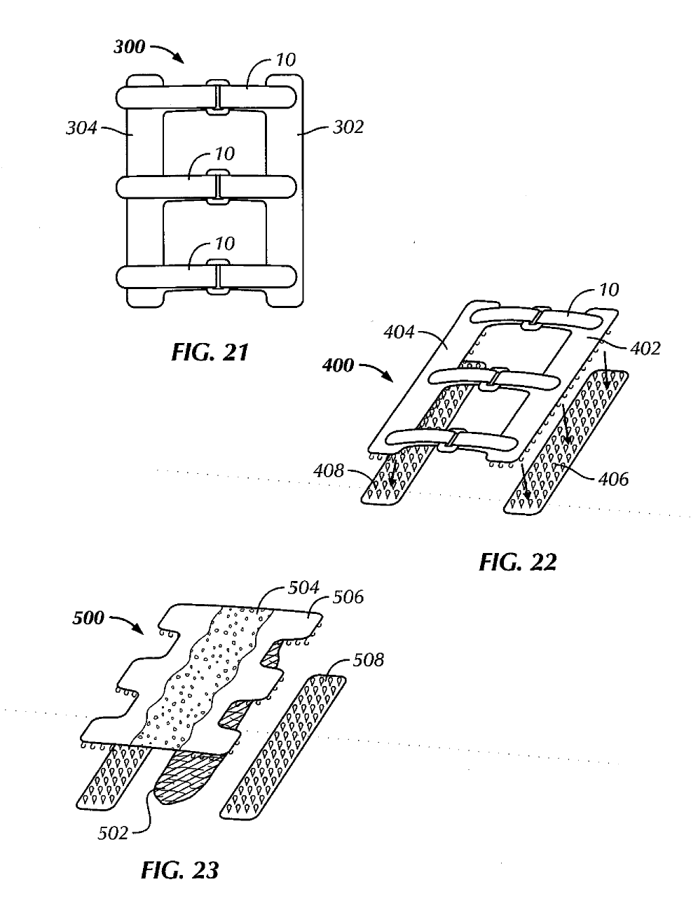

FIG. 21 shows a strap system 300 in which plural strap assemblies 10 can be disposed parallel to each other and spaced from each other transversely as shown. The adhesive pads of each assembly 10 are engaged with opposed parallel footings 302, 304, essentially flexible, relatively long base strips with adhesive on their bottom surfaces to engage the skin, which distributes the tensile load over a larger area of skin. The footings 302, 304 can support various numbers of strap assemblies for connecting purposes. Convenient increments can be provided and can also be customized by trimming off unneeded straps.

FIG. 22 is an exploded perspective view of another embodiment of a strap system 400 similar to that shown in FIG. 21 except that the footings 402, 404 do not have adhesive bottom surfaces. Instead, the bottom surfaces of the footings include hook (or loop) fasteners and engage complementarily-configured loops (or hooks) on the top surfaces of respective replaceable anchors 406, 408. As shown, the anchors 406, 406 may be substantially the same shape and size as the footings 402, 404. The bottom surfaces of the anchors 406, 408 have adhesive disposed on them to adhere to the skin of the patient. Once the adhesive on the anchors loses its anchoring strength, the footings with strap assemblies system can be detached from the anchors, new adhesive anchors applied to the patient, then the footings with strap assemblies reattached to the new anchors.

FIG. 23 is an exploded view of a flexible tension pad 500 that can be placed over a wound 502 to cover the wound. A wound dressing 504 can be disposed on the surface of the pad 500 facing the wound 502. Opposed left and right sides of the pad 500 may be formed with plural arms 506 which extend laterally outward and which have bottom surfaces with adhesive on them, so that a person can position the pad 500 over the wound 502, grasp the arms 506 on the opposing left and right sides of the pad, pull the arms outwardly to tension the pad, and then lower the pad onto the patient to stick the pad onto the skin in a tensioned state. Or, in the embodiment shown, instead of adhesive on the arms 506, adhesive footings 508 may be provided and the arms adhered to the footings 508 with, e.g., hook and loop fasteners according to principles described above.

FIG. 24 shows a flexible and hence conformed adhesive anchor base 600 that conforms to the part of the body on which it is attached, a heel being shown as an example. An opening 602 in the base fits over the wound. One or more strap assemblies 10 may be engaged with the base 600 using, e.g., hook and loop fasteners and then the strap assemblies 10 tensioned using any of the buckles described above. This approach can be applied to other unusual shaped areas of the body.

Strap material that may be used includes mesh, plastic, or other flexible inelastic preferably biocompatible material. Buckle/connector material may include plastic, preferably relatively soft yet rigid plastic, or less preferably metal.

While the particular DESIGNS FOR WOUND SUPPORT APPARATUS are herein shown and described in detail, it is to be understood that the subject matter which is encompassed by the present invention is limited only by the claims. Components included in one embodiment can be used in other embodiments in any appropriate combination. For example, any of the various components described herein and/or depicted in the Figures may be combined, interchanged or excluded from other embodiments.

“A system having at least one of A, B, and C” (likewise “a system having at least one of A, B, or C” and “a system having at least one of A, B, C”) includes systems that have A alone, B alone, C alone, A and B together, A and C together, B and C together, and/or A, B, and C together, etc.

What is claimed is:

1. Assembly comprising:
a first strap arrangement having a first end configured for engaging skin of a patient and a second end, a first strap body extending between the ends of the first strap arrangement;
a second strap arrangement having a first end configured for engaging skin of a patient and a second end, a second strap body extending between the ends of the second strap arrangement; and
a quick release buckle configured for engaging the strap bodies to hold the strap bodies in tension.

2. The assembly of claim 1, wherein the quick release buckle comprises first and second apertures separated from each other by a central wall, each aperture being defined at least in part by the central wall and a respective outer wall spaced from the central wall and generally parallel thereto, at least one of the outer walls being discontinuous to form a passageway through which a strap body can be passed to engage and disengage the strap with the buckle.

3. The assembly of claim 2, wherein both outer walls are discontinuous and form respective passageways, the buckle being flexible such that a person can compress a first one of the discontinuous outer walls to thereby enlarge the passageway formed by a second one of the outer walls.

4. The assembly of claim 1, wherein the buckle comprises a female element with an open end and side slots and a male element with opposed resilient arms, the tangs of the male element being receivable through the open end of the female element and each being exposable through a respective slot, so that a person can slide the male element into the female element and the tangs engage the slots to hold the elements together, and so that a person can press the tangs exposed in the respective slots toward each other and pull the elements apart to disengage the elements.

5. The assembly of claim 1, wherein the first ends of the respective strap arrangements include respective pads engaged with the respective strap bodies, each pad having an adhesive surface for adhering to the skin and an opposed receiving surface configured for engaging the respective second end of the respective strap arrangement.

6. The assembly of claim 1, wherein the first ends of the respective strap arrangements include respective pads configured to be held onto the skin by staples or sutures or subcutaneous anchors.

7. The assembly of claim 1, wherein at least one end of at least one of the strap arrangements includes at least one visible reference line oriented perpendicularly to a long axis defined by the strap arrangement.

8. The assembly of claim 1, wherein the assembly is a first assembly defining a long axis and engaged with opposed elongated footings oriented perpendicularly to the long axis, the footings also engaged with at least a second assembly oriented parallel to and space from the first assembly.

9. The assembly of claim 8, wherein the footings are configured to engage respective skin adhesive pads.

10. The assembly of claim 1, comprising a wound dressing constituent on the buckle.

11. The assembly of claim 1, wherein the buckle is curved in longitudinal cross-section.

12. Assembly comprising:
a first strap arrangement having a first end configured for engaging skin of a patient and a second end, a first strap body extending between the ends of the first strap arrangement;
a second strap arrangement having a first end configured for engaging skin of a patient and a second end, a second strap body extending between the ends of the second strap arrangement; and
a connector configured for engaging both strap bodies to hold the strap bodies in tension, the connector including at least one tension indicator.

13. The assembly of claim 12, wherein the tension indicator includes at least one light emitting diode (LED) energized responsive to a predetermined tension being reached.

14. The assembly of claim 12, wherein the tension indicator includes at least one digital display presenting an alpha-numeric indication of tension.

15. The assembly of claim 12, wherein the connector includes a first piece engaged with the first strap body and a second piece reciprocatingly engaged with the first piece and engaged with the second strap body, a spring interconnecting the pieces, the tension indicator including at least one transverse hash mark on at least one of the pieces.

16. The assembly of claim 12, wherein the tension indicator includes at least one wireless transceiver generating a wireless signal representative of tension.

17. The assembly of claim 12, wherein the first ends of the respective strap arrangements include respective pads engaged with the respective strap bodies, each pad having an adhesive surface for adhering to the skin and an opposed receiving surface configured for engaging the respective second end of the respective strap arrangement.

18. The assembly of claim 12, wherein the first ends of the respective strap arrangements include respective pads configured to be held onto the skin by staples or sutures or subcutaneous anchors.

19. The assembly of claim 12, wherein at least one end of at least one of the strap arrangements includes at least one visible reference line oriented perpendicularly to a long axis defined by the strap arrangement.

20. The assembly of claim 12, wherein the assembly is a first assembly defining a long axis and engaged with opposed elongated footings oriented perpendicularly to the long axis, the footings also engaged with at least a second assembly oriented parallel to and space from the first assembly.

21. The assembly of claim 20, wherein the footings are configured to engage respective skin adhesive pads.

22. The assembly of claim 12, comprising a wound dressing constituent on the connector.

23. The assembly of claim 12, wherein the connector is curved in longitudinal cross-section.

24. Assembly comprising:
a first strap arrangement having a first end configured for engaging skin of a patient and a second end, a first strap body extending between the ends of the first strap arrangement;
a second strap arrangement having a first end configured for engaging skin of a patient and a second end, a second strap body extending between the ends of the second strap arrangement; and
a connector configured for engaging both strap bodies to hold the strap bodies in tension, the connector including a first end portion engaged with the first strap body, a second end portion engaged with the second strap body, and an interconnect connecting the first and second end portions, the interconnect being selected from the group of magnet, elastic band, arcuate flexure.

25. The assembly of claim 24, wherein the first end portion defining a first aperture through which the first strap body extends, the second end portion defining a second aperture through which the second strap body extends, and the interconnect includes at least a first magnet engaged with the first end portion and at least a ferromagnetic element engaged with the second end portion.

26. The assembly of claim 24, wherein the first end portion defines a first aperture through which the first strap body extends, the second end portion defines a second aperture through which the second strap body extends, and the interconnect includes an elastic band.

27. The assembly of claim 24, wherein the interconnect is an arcuate flexure having opposed first and second ends defining the first and second end portions, respectively.

28. The assembly of claim 24, wherein the first ends of the respective strap arrangements include respective pads engaged with the respective strap bodies, each pad having an adhesive surface for adhering to the skin and an opposed receiving surface configured for engaging the respective second end of the respective strap arrangement.

29. The assembly of claim 24, wherein the first ends of the respective strap arrangements include respective pads configured to be held onto the skin by staples or sutures or subcutaneous anchors.

30. The assembly of claim 24, wherein at least one end of at least one of the strap arrangements includes at least one visible reference line oriented perpendicularly to a long axis defined by the strap arrangement.

31. The assembly of claim 24, wherein the assembly is a first assembly defining a long axis and engaged with opposed elongated footings oriented perpendicularly to the long axis, the footings also engaged with at least a second assembly oriented parallel to and space from the first assembly.

32. The assembly of claim 31, wherein the footings are configured to engage respective skin adhesive pads.

33. The assembly of claim 24, comprising a wound dressing constituent on the connector.

34. The assembly of claim 24, wherein the connector is curved in longitudinal cross-section.
